(19) United States
(12) Patent Application Publication
(10) Pub. No.: US 2024/0415837 A1
(43) Pub. Date: Dec. 19, 2024

(54) PHARMACOLOGIC MODULATION OF 5-FLUOROURACIL BY FOLINIC ACID AND VITAMIN B6 FOR THE TREATMENT OF PATIENTS WITH CARCINOMA

(71) Applicant: David MACHOVER, PARIS (FR)

(72) Inventor: David MACHOVER, PARIS (FR)

(21) Appl. No.: 18/702,490

(22) PCT Filed: Oct. 17, 2022
(86) PCT No.: PCT/EP2022/078836

§ 371 Date: Apr. 18, 2024

(30) Foreign Application Priority Data
Oct. 19, 2021 (FR) 2111099

(51) Int. Cl. A61K 31/513; A61K 31/282; A61K 31/4415; A61K 31/4745; A61K 31/519; A61P 35/04

(52) U.S. Cl. CPC A61K 31/513; A61K 31/282; A61K 31/4415; A61K 31/4745; A61K 31/519; A61P 35/04

(57) ABSTRACT

An antitumor pharmaceutical composition including (i) a fluoropyrimidine, (ii) a B6 vitamer, and (iii) a folate for its use in the treatment of cancer in a subject in need thereof. The antitumor pharmaceutical composition may be for a simultaneous, separate, or sequential use for the treatment of cancer, and may be administered sequentially or concomitantly with one or more immunotherapeutic, chemotherapeutic or radiotherapeutic agent.

### SUMMARY OF THE INVENTION

High concentration pyridoxal 5′-phosphate, the cofactor of vitamin B6, potentiates cytotoxicity in cancer cells exposed to 5-fluorouracil (5-FU or FUra) and folinic acid (FA). We studied the effect of high-dose pyridoxine (PN) on antitumor activity of regimens comprising FUra and FA in 27 advanced breast carcinoma patients. Of 18 previously untreated patients, 12 had tumors that did not overexpress HER2 (Group I), and 6 that overexpressed HER2 (Group II). Nine patients (Group III) had prior chemotherapy. Group I received FAC (doxorubicin, cyclophosphamide, FUra, FA) or AVCF (doxorubicin, vinorelbine, cyclophosphamide, FUra, FA) followed by TCbF (paclitaxel carboplatin, FUra, FA). Groups II, and III received TCbF. Pyridoxine iv (1,000-3,000 mg/day) preceded each FA and FUra. Group II also received trastuzumab and pertuzumab. 26 patients responded. Three patients in Group I had CRs and 9 had PRs with 62%-98% reduction rates; 4 patients in Group II had CRs and 2 had PRs with 98% reduction. Of 7 measurable patients in Group III, 2 attained CRs, and 5 had PRs with 81-94% reduction rates. Median time to response was 3.4 months. Unexpected toxicity did not occur. This pilot study suggests that high-dose vitamin B6 enhances antitumor potency of regimens comprising FUra and FA.

### PRIOR ART

Fluorodeoxyuridine monophosphate (FdUMP), the active metabolite of 5-fluorouracil (FUra), binds to thymidylate synthase (TS) and the folate cofactor N5-N10 methylene tetra hydro pteroylglutamate (CH₂—H₄PteGlu) to form a TS-inactivating [FdUMP-TS-CH₂—H₄PteGlu] ternary complex, whose dissociation decreases as CH₂—H₄PteGlu is augmented over a wide concentration range up to levels greater than 1 mM.¹⁻³ Exposure of cancer cells to fluorodeoxyuridine with high concentration N5-formyl tetra hydro pteroylglutamate [5-HCO-H₄PteGlu; folinic acid (FA); leucovorin] up to 20 μM in vitro resulted in formation of greater amounts of ternary complex than with the single fluoropyrimidine leading to gradual enhancement of the cytotoxic effect.⁴ FdUMP-mediated TS inhibition prevents synthesis of thymidine triphosphate (dTTP) leading to deoxy nucleotide triphosphate (dNTP) pool imbalance, and results in accumulation of deoxy uridine triphosphate (dUTP) and fluorodeoxyuridine triphosphate (FdUTP), which lead to genomic DNA replication defects including DNA mismatch and altered replication fork progression eliciting DNA damage cell responses and, ultimately, cell death.⁵⁻⁸

Translation of these pharmacologic principles to the clinics led to regimens of FUra combined with high dose FA possessing greater antitumor efficacy than single FUra that are currently used for treatment of patients with colorectal, gastric, and pancreas adenocarcinomas.^{9,10} However, further attempts at improvement of the anticancer effect of the modulation did not convincingly succeed. Probably, the effect of the combination has reached a limit that could not be overcome by using higher doses of folate of any type or through changes in modalities of administration of the compounds.

Effectiveness of the modulation of fluoropyrimidines by folates varies among cancer cells. Variation was related to differences in folate polyglutamationn¹¹ and to capacities for expansion of intracellular CH₂—H₄PteGlu pools. Intracellular CH₂—H₄PteGlu concentration changes resulting from exposure to reduced folates were previously studied.¹¹⁻²⁰ From reported data, it is unlikely that supplementation of cancer cells with any amount of folate would result in durable rise of CH₂—H₄PteGlu up to concentration levels required to increase the tightness of FdUMP binding to TS for maximum stability of the ternary complex.^{2,3} In most studies, only limited increase of CH₂—H₄PteGlu concentration up to levels far below that required for optimum stabilization of the ternary complex occurred, followed by rapid decline after discontinuation of folate exposure.¹¹⁻²⁰ One explanation for these findings is the rapid turnover of folates in cancer cells²¹ including the irreversible reduction of CH₂—H₄PteGlu to N5-methyl tetra hydro pteroylglutamate (CH₃-H₄PteGlu) (FIG. 1). Poor expansion of CH₂—H₄PteGlu pools in cancer cells may also result from insufficient production of this folate. Synthesis of CH₂—H₄PteGlu from tetra hydro pteroylglutamate (H₄PteGlu) results from two pathways. One is the transfer of Cβ of serine to H₄PteGlu with formation of glycine and CH₂—H₄PteGlu catalyzed by serine hydroxymethyl transferase (SHMT), a ubiquitous pyridoxal 5′-phosphate (PLP)-dependent enzyme that is the major source of one-carbon units for cellular metabolism.²²⁻²⁵ The second pathway is the Glycine Cleavage System that catalyzes glycine cleavage up to formation of CH₂—H₄PteGlu in mitochondria.^{26,27} The rationale for studies aiming at modulate the fluoropyrimidines by reduced folates and vitamin B6 in tandem lies in the low affinity for binding of SHMT apoenzyme to cofactor. SHMT from various mammalian sources including man was found to bind to cofactor with K_{d} from 250 nM to as high as 27 μM,²²⁻²⁵ while naturally occurring PLP levels in erythrocytes vary approximately from 30 to 100 nmol/L of cells,^{27,28} which indicates that SHMT activity should be sensitive to intracellular PLP concentration modifications. Availability of vitamin B6 translates into significant intracellular folate-mediated one-carbon metabolism changes. The human mammary MCF-7 carcinoma cells cultured in pyridoxine deficient medium exhibited decrease in PLP levels, SHMT activity, and S-adenosyl methionine levels. Addition of PLP to these cells greatly increased SHMT activity from that measured in cells grown in pyridoxine (PN)-deficient medium.²⁴ Dietary vitamin B6 deficiency in rats reduced PLP levels and SHMT activity in liver, which were accompanied by decreased remethylation of homocysteine to methionine with methyl groups from serine i.e., resulting from decreased SHMT-catalyzed synthesis of CH₂—H₄PteGlu, and subsequently of CH₃—H₄PteGlu, a cofactor of methionine synthase (FIG. 1).²⁹

From these data we assumed that, in tumors, naturally occurring PLP levels are too small to allow intracellular SHMT-dependent conversion of H₄PteGlu into CH₂—H₄PteGlu in amounts required to improve inhibition of TS by FdUMP by increasing stability of the ternary complex.³⁰ To test for variations of SHMT activity resulting from PLP level changes in cancer cells, we conducted experiments in the human colon carcinoma HT29, and HCT116 cell lines, and in the murine leukemia L1210 cell line in vitro to investigate for interactions between FUra, FA, and PLP on cell growth.³⁰ Supplementation of cancer cells exposed to FUra with high concentrations of PLP and FA in tandem strongly potentiated the cytotoxic activity of FUra in the three cell lines and resulted in powerful growth inhibiting synergistic interaction in HT29 and in L1210 cells, while summation was found in HCT116 cells. These findings support the hypothesis of expansion of CH₂—H₄PteGlu pools resulting from increase in SHMT activity by supplying cancer cells with PLP.

Intracellular pharmacokinetic experiments were conducted in mice to study the physiologic capacities for the biochemical modulation of FUra by vitamin B6 to be achieved in vivo by expanding intracellular PLP pools, and for possible limitations.³⁰ BALB/c mice were given high doses of pyridoxamine (PM) or pyridoxine (PN) at 450 mg/kg by intraperitoneal route at time 0 only (t0), or twice at time 0 and after 12 hours from start (i.e., at times t0 and t12h) before being sampled at regular intervals. Studies determined that erythrocyte levels of PLP after parenteral administration of each unphosphorylated B6 vitamers rose to concentration levels within the range of K_{d} values of SHMT binding to cofactor,²²⁻²⁵ and that newly synthesized PLP was rapidly cleared from cells.³⁰ Levels decreased to reach baseline concentrations by 12 hours after injection, with no measurable cumulative effect when administrations of B6 vitamer were repeated at 12-hour interval. Rapid decline of intracellular PLP levels after vitamin B6 administration was also reported in man.^{27,28} From these data, we thought that administration of high-dose unphosphorylated B6 vitamer to patients treated with FUra and FA would increase intracellular PLP levels within tumors, leading to augmentation of CH₂—H₄PteGlu synthesis resulting in long-term TS inhibition and enhanced antitumor effect. Additional analysis of data obtained from these experiments³⁰ determined that intraerythrocytic PLP peak concentration levels, and PLP area under the concentration vs time curve in 12 hours from injection (AUCₜ₀₋ₜ₁₂ₕ in nmol PLP-12 hour/L cells) in mice that received intraperitoneal PM were 3.7- and 6.7-fold greater than that measured in animals having received PN, respectively (FIG. 2). An explanation for the significant discrepancy of intracellular pharmacokinetics between these unphosphorylated B6 vitamers could lie in differences for conversion in cofactor. Both PM and PN are phosphorylated by the ATP-dependent pyridoxal kinase (PLK) in PMP and PNP respectively, and then oxidized to PLP by the flavin mononucleotide (FMN)-dependent pyridoxine (pyridoxamine) 5′-phosphate oxidase (PNPOx),³¹⁻³⁴ whose affinity for PMP was reported greater than that for PNP; measured Kₘ of PNPOx for PMP, and for PNP were 1.0 μM, and 1.8 μM, respectively.³⁴ In addition to this catalytic sequence that is common to both vitamers, PMP was described to be reversibly converted in PLP through a pyridoxamine aminotransferase-catalyzed pathway.³¹⁻³³ Further studies are required to explore the findings presented herein which indicate that parenteral PM may possess an advantage over PN to expand intracellular PLP pools.

Chemotherapy regimens for treatment of patients with breast carcinoma, mostly combining two to four cytostatics, often administered in sequence, include anthracyclines, taxanes, vinca alkaloids, alkylating agents, platinum coordination compounds, and fluoropyrimidines. Currently used standard regimens produce effective but limited antitumor effect in patients with tumors in advanced stages whose response rates range between 40 and 60 percent, with approximately 10% of patients attaining a complete response.^{35,36} Phase II studies of FUra and folinic acid administered either as single agents or in combination with a second cytostatic to patients with advanced breast carcinoma led to favorable results.³⁷⁻⁴⁴ Response rates reported in these studies ranged from 36% to 41% in patients treated with FUra and folinic acid as single agents, and from 28% to 70% in patients who received FUra, and FA combined with paclitaxel or vinorelbine. However, up to now FUra plus FA modulation-based schemas have not been widely recognized as components of first-line treatment options for breast carcinoma.

We report herein a translational pilot study for patients with breast carcinoma in advanced stages who were not amenable to resection or radiotherapy with curative intent, and whose standard treatment regimens included a combination of FUra and FA, consisting in addition of pyridoxine (PN) in high dose to these combination regimens. Pyridoxine in high doses used for treatment of various conditions in man was reported to be safe, although it caused sensory peripheral neuropathy when it was administered in extremely high doses for long periods of time.^{45,46} From these prior data, we thought that vitamin B6 administered in short-time courses followed by drug-free intervals in doses far below that reported to be toxic in man, was not likely to expose patients to increased risk of neuropathy. However, during the course of the study we proceeded with progressive dose escalation of pyridoxine and were particularly cautious on neurologic signs and symptoms.

Modulation of FUra by high-dose FA and PN in tandem was used in a pilot study for treatment of patients with unresectable or metastatic colorectal adenocarcinoma, pancreas adenocarcinoma, and squamous cell carcinoma of the esophagus.⁴⁷ Addition of high-dose PN to standard regimens of chemotherapy comprising FUra and FA led to high rate of antitumor responses of early onset and great magnitude with no detrimental effect on toxicity from that expected using these same regimens in absence of PN.

The present study included 27 patients with breast adenocarcinoma in advanced stage who were treated from December 2014 to February 2021 in a single clinical center. Patients presented either with highly advanced loco regional tumor involvement including inflammatory carcinoma accompanied or not with distant nodal, bone and/or visceral metastases, or with distant metastases only. We used a casuistic analytic approach owing to the limited number of patients entered, and the great extent of tumor at inclusion.

### DETAILED DESCRIPTION

The object of the invention is an antitumor pharmaceutical composition comprising (i) a fluoropyrimidine, (ii) a B6 vitamer, and (iii) a folate for its use in the treatment of breast cancer in a subject in need thereof.

The term “vitamin B6” as used in this application refers to any compound or mixture of compounds that exhibit any biological activity in any vitamin B6 bioassay. B6 vitamins include, but are not limited to, pyridoxine (also known as pyridoxol or PN), pyridoxal (PL), pyridoxamine (PM), the 5′-phosphorylated derivatives of any of the above three compounds, namely pyridoxine 5′-phosphate (PNP), pyridoxamine 5′-phosphate (PMP) and pyridoxal 5′-phosphate (PLP), their acetic esters, their pharmaceutically acceptable salts and any related derivatives or compounds which can be converted to PLP, PNP or PMP in a tested organism.

The antitumor pharmaceutical composition according to the invention can for its use in the treatment of breast cancer in a subject in need thereof, be administered in a simultaneous, separate or sequential way.

In the antitumor pharmaceutical composition according to the invention for its use in the treatment of breast cancer in a subject in need thereof, the fluoropyrimidine is selected from the group consisting of 5-fluorouracil, capecitabine, 5-fluoro-2′-deoxyuridine, ftorafur, emitefur, eniluracil/5-FU, S-1, UFT and mixtures thereof.

In the antitumor pharmaceutical composition according to the invention for its use in the treatment of breast cancer in a subject in need thereof, the fluoropyrimidine is preferably the 5-fluorouracil.

In the antitumor pharmaceutical composition according to the invention for its use in the treatment of breast cancer in a subject in need thereof the B6 vitamer is preferably selected from the group consisting of pyridoxine, pyridoxal, pyridoxamine, 5′-phosphorylated derivatives thereof, acetate esters thereof, pharmaceutically compatible salts thereof, and mixtures thereof.

In the antitumor pharmaceutical composition according to the invention for its use in the treatment of breast cancer in a subject in need thereof the B6 vitamer is preferably selected from the group consisting of pyridoxine and pyridoxamine.

The invention has more particularly for its object an antitumor pharmaceutical composition according to the invention for its use in the treatment of breast cancer in a subject in need thereof wherein the B6 vitamer is administered at a dose high enough to achieve plasma or intracellular levels of PLP equal to or greater than those required for optimal synergistic effect of fluoropyrimidines.

In the antitumor pharmaceutical composition according to the invention for its use in the treatment of breast cancer in a subject in need thereof, the folate is selected from the group consisting of folic acid, dihydrofolate, tetrahydrofolate, 5-methyltetrahydrofolate, 5,10-methylenetetrahydrofolate (MTHF), 5-formyltetrahydrofolate, 5,10-methenyltetrahydrofolate, 10-formyltetrahydrofolate, 5-formiminotetrahydrofolate, [6RS]-5-formyltetrahydrofolate and the active stereoisomers of any of these folates, [6S]-5-formyltetrahydrofolate (levofolinate), [6R]-5,10-methylenetetrahydrofolate ([6R]-MTHF), the pharmaceutically compatible salts including the hemisulfate salts of these folates and mixtures thereof. One can in particular cite the hemisulfate salt of [6R]-MTHF (arfolitixorin, Modufolin®) and the calcium or sodium salts of levofolinate.

In the antitumor pharmaceutical composition according to the invention for its use in the treatment of breast cancer in a subject in need thereof, the folate is selected from the group consisting most preferably of 5-formyltetrahydrofolate or [6S]-5-formyltetrahydrofolate, the hemisulfate salt of [6R]-MTHF (arfolitixorin, Modufolin®) or the calcium or sodium levofolinate. The preferred levofolinate salt is the calcium salt.

The invention has more particularly for its object an antitumor pharmaceutical composition comprising (i) a fluoropyrimidine, (ii) a B6 vitamer, and (iii) a folate for its use in the treatment of breast cancer in a subject in need thereof wherein said composition is administered sequentially or concomitantly with one or more immunotherapeutic, chemotherapeutic or radiotherapeutic agent.

The word “patient” as used in this application refers to a male or female person. As female breast cancers are much more common than male breast cancers, the invention is specifically directed to female breast cancer patients.

The conditions under which the various constituents of the pharmaceutical compositions are administered for their use according to the invention can be summarised as follows: When administered intravenously, fluoropyrimidines may be used in the context of the invention by bolus administration, by short term intravenous infusion which may be over a period of, for example, one to 12 hours, by continuous infusion, or by a mixture of these methods. Typically, a bolus of FUra may be administered to a subject at a dose of 370-500 mg/m2 per day for 5 days every 3 to 5 weeks, or 500 mg/m2 per week. Alternatively, one or more doses of FUra may be administered by continuous infusion over a period of at least 22 hours per dose. Continuous administration of FUra may include an intravenous bolus dose of 400 mg/m2 followed by 600 mg/m2 over 22 hours for two days. Alternatively, a bolus dose of 400 mg/m2 of FUra may be followed by a dose of 2400 mg/m2 administered over 46 hours. The doses of vitamin B6 conventionally used to treat vitamin B6 deficiency are well known to the skilled person. Typically, conventional doses of vitamin B6 used to treat a vitamin B6 deficiency are from 1.3 to 300 mg/day, more particularly from 50 to 300 mg/day. In particular, the B6 vitamins used in the context of the invention are administered at a dose at least two times higher, in particular at least three times higher, at least four times higher, at least five times higher, at least six times higher, at least seven times higher, at least eight times higher, at least nine times higher, at least ten times higher, at least twenty times higher, at least thirty times higher, at least forty times higher or at least fifty times higher than the dose conventionally used to treat vitamin B6 deficiency. Even higher doses of vitamin B6 can also be used in the context of the invention. In particular, the B6 vitamins used in the invention (e.g., PN, PL, PM or any of their 5′-phosphorylated derivatives) are administered at a high dose (and/or various modes of administration—i.e., rapid iv injection; short iv infusions; or continuous iv infusion of variable duration) to achieve plasma and/or intracellular levels of PLP equal to or greater than those required for an optimal synergistic effect of fluoropyrimidines, typically equal to or greater than 160 μmol/L.

The folates used in the invention may be administered orally or parenterally, in particular orally, intravenously, intramuscularly or subcutaneously. When administered intravenously, folates may be used in the invention by bolus administration, by continuous infusion or by a mixture of both. Typically, folates, in particular folinic acid, may be administered in a dose ranging from 20 to 1000 mg/m2/day, more particularly in a dose ranging from 25 to 500 mg/m2/day, in particular in a dose ranging from 50 to 400 mg/m2/day, even more particularly in a dose ranging from 100 to 200 mg/m2/day. In a particular embodiment, folates, in particular folinic acid, may be administered at a low dose, i.e., at a dose below 25 mg/m2/day. Alternatively, folates, in particular folinic acid, may be administered at a high dose, i.e., at a dose above 200 mg/m2/day. In particular, folates, especially folinic acid, may be administered at a high dose to allow a therapeutically effective plasma concentration, i.e., a plasma concentration of 10 mM.

The invention has also for its object an antitumor pharmaceutical composition comprising (i) a fluoropyrimidine, (ii) a B6 vitamer, and (iii) a folate for its use in the treatment of cancer, in particular colorectal cancer including advanced and metastatic colorectal cancer and pancreas adenocarcinomas, in a subject in need thereof, said treatment comprising the following steps:
a) On Day 1, administering over a period of 10-30 minutes, preferably about 15 minutes, an IV bolus containing 100-1000 mg/m², preferably 200 mg/m² of a folate, preferably levofolinate calcium or arfolitixorin followed by:
b) administering over a period of 10-30 minutes, preferably about 15 minutes, an IV bolus, containing a dose higher than 3000 mg of a B6 vitamer preferably pyridoxine and pyridoxamine followed by
c) administering an IV bolus containing 100-1000 mg/m², preferably about 600 mg/m² of 5-FU over a period of 2 hours followed by
d) administering an IV infusion containing 100-500 mg/m², preferably about 350 mg/m² of 5-FU over a period of 22 hours,

Said steps a) to d) are repeated on Day 2,

wherein, optionally
1) On day 1, said steps a) to d) are preceded by
   i) administering over a period of 30-180 minutes, preferably 120 minutes an IV bolus containing 50-100 mg/m², preferably about 85 mg/m² of oxalato-platinum (1-OHP) optionally followed by
   ii) administering over a period of 10-60 minutes, preferably about 30 minutes an IV bolus containing 100-200 mg/m², preferably about 180 mg/m² of camptothecin-11 (CPT11)
   and/or
2) On day 1 and on Day 2, between said steps c) and d) administering over a period of 10-30 minutes, preferably about 15 minutes, an IV bolus, containing a dose higher than 3000 mg of a B6 vitamer preferably pyridoxine and pyridoxamine.

As indicated above, on day 1 of the treatment steps a) to d) can be preceded by the administration of an IV bolus of oxalato-platinum (1-OHP) optionally followed by the administration of an IV bolus of camptothecin-11 (CPT11).

The optional administration of a platinum derivative (preferably oxalato-platinum (1-OHP) and/or a camptothecin derivative (preferably CPT 11) before the administration of a fluoropyrimidine, a B6 vitamer and a folate is more particularly adapted to the treatment of digestive cancers like colorectal cancer including advanced and metastatic colorectal cancer and pancreas adenocarcinomas.

When the treatment of another type of cancer, in particular breast cancer is contemplated, anticancer products like anthracyclins, in particular Daunorubicin, Doxorubicin, Epirubicin, Idarubicin, Mitoxantrone, Pirarubicin or Pixantrone can be administered in addition the administration of a fluoropyrimidine, a B6 vitamer, and a folate. Other anticancer products that can be administered in addition to a fluoropyrimidine, a B6 vitamer, and a folate in the treatment of breast cancer include taxanes, preferably paclitaxel, nab-paclitaxel, or docetaxel; vinca alkaloids like Vinblastine, vinorelbine (navelbine), vincristine and vindesine; Cyclophosphamide or platinum-derived compounds, preferably carboplatin.

The invention has also for its object an antitumor pharmaceutical composition comprising (i) a fluoropyrimidine, (ii) a B6 vitamer, and (iii) a folate for its use in the treatment of cancer, in particular breast cancer, in a subject in need thereof, said treatment comprising the following steps:
a) On Day 1, administering an IV bolus of a folate, preferably levofolinate calcium or arfolitixorin followed by:
b) administering an IV bolus, containing a B6 vitamer preferably pyridoxine and pyridoxamine followed by
c) administering a IV bolus containing 5-FU over a period of 2 hours followed by
d) administering an IV infusion containing of 5-FU
and, optionally,
e) Said steps a) to d) are repeated on Days 2 to 4,
wherein,
On day 1, said steps a) to d) are preceded by the administration of an anthracyclin, in particular Daunorubicin, Doxorubicin, Epirubicin, Idarubicin, Mitoxantrone. Pirarubicin or Pixantrone and/or a taxane, preferably paclitaxel, nab-paclitaxel, or docetaxel and/or a vinca alkaloid like Vinblastine, vinorelbine (navelbine), vincristine and/or vindesine; Cyclophosphamide and/or platinum-derived compounds, preferably carboplatin.

The invention has more particularly for its object an antitumor pharmaceutical composition comprising (i) a fluoropyrimidine, (ii) a B6 vitamer, and (iii) a folate for its use according to the above in the treatment of cancer, in particular colorectal cancer including advanced and metastatic colorectal cancer and pancreas adenocarcinomas, in a subject in need thereof, said treatment being administered each 14 days. The two-day protocol is repeated every 14 days for as long as an optimal anti-tumoral effect is achieved. An optimal anti-tumoral effect can be defined as a tumour regression or at least as a plateauing of the tumour size.

By a dose higher than 3000 mg of a B6 vitamer is meant a dose that produces the optimal biological effect (Optimal Biological Dose or OBD). High to very high doses of a B6 vitamer can be advantageously used in the implementation of the invention. Such high or very high doses of a B6 vitamer can be in the order of 3000, 6000, 12000,18000 mg or more.

The invention has more particularly for its object an antitumor pharmaceutical composition comprising (i) a fluoropyrimidine, (ii) a B6 vitamer, and (iii) a folate for its use according to claim 10 in the treatment of cancer in a subject in need thereof, wherein the cancer is selected from the bladder, blood, bone, bone marrow, brain, breast, colon, esophagus, gastrointestinal, gum, head, kidney, liver, lung, nasopharynx, neck, ovary, prostate, skin, stomach, testis, tongue, pancreatic or uterus cancer in particular colorectal cancer including advanced and metastatic colorectal cancer and pancreas adenocarcinomas.

In a preferred embodiment, the invention can be performed every 14 days according to the following non-limitative ways:

### Schema 1

### On Day 1,

1) administering over a period of about 120 minutes an IV bolus containing about 85 mg/m2 of oxalato-platinum (1-OHP, oxaliplatin) followed by
2) administering over a period of about 30 minutes an IV bolus containing about 180 mg/m² of camptothecin-11 (CPT11, Irinotecan) followed by
3) administering over a period of about 15 minutes, an IV bolus containing about 200 mg/m2 of a folate, selected from levofolinate and arfolitixorin followed by
4) administering over a period of 10-30 minutes, preferably about 15 minutes, an IV bolus, containing a dose higher than 3000 mg of a B6 vitamer preferably pyridoxine and pyridoxamine followed by
5) administering an IV bolus containing about 625 mg/m² of 5-FU over a period of about 2 hours optionally followed by
6) administering an IV infusion containing about 375 mg/m² of 5-FU over a period of 22 hours,

### On Day 2,

1) administering over a period of about 15 minutes, an IV bolus containing about 200 mg/m² of a folate, selected from levofolinate and arfolitixorin followed by
2) administering over a period of 10-30 minutes, preferably about 15 minutes, an IV bolus, containing a dose higher than 3000 mg of a B6 vitamer preferably pyridoxine and pyridoxamine followed by
3) administering an IV bolus containing about 625 mg/m² of 5-FU over a period of about 2 hours optionally followed by
4) administering an IV infusion containing about 375 mg/m² of 5-FU over a period of 22 hours,

### Schema 2:

### On Day 1,

1) administering over a period of about 120 minutes an IV bolus containing about 85 mg/m2 of oxalato-platinum (1-OHP, oxaliplatin) followed by
2) administering over a period of about 30 minutes an IV bolus containing about 180 mg/m² of camptothecin-11 (CPT11, Irinotecan) followed by
3) administering over a period of about 15 minutes, an IV bolus containing about 200 mg/m2 of a folate selected from levofolinate and arfolitixorin followed by
4) administering over a period of 10-30 minutes, preferably about 15 minutes, an IV bolus, containing a dose higher than 3000 mg of a B6 vitamer preferably pyridoxine and pyridoxamine followed by
5) administering an IV bonus containing about 625 mg/m² of 5-FU over a period of about 2 hours optionally followed by
6) administering over a period of 10-30 minutes, preferably about 15 minutes, a second IV bolus, containing more than 3000 mg of a B6 vitamer preferably pyridoxine and pyridoxamine followed by
7) administering a continuous infusion containing about 375 mg/m² of 5-FU over a period of 22 hours,

### On Day 2,

1) administering over a period of about 15 minutes, an IV bolus containing about 200 mg/m² of a folate selected from levofolinate and arfolitixorin followed by 2) administering over a period of 10-30 minutes, preferably about 15 minutes, an IV bolus, containing a dose higher than 3000 mg of a B6 vitamer preferably pyridoxine and pyridoxamine followed by
3) administering an IV bolus containing about 625 mg/m² of 5-FU over a period of about 2 hours optionally followed by
4) administering over a period of 10-30 minutes, preferably about 15 minutes, a second IV bolus, containing a dose higher than 3000 mg of a B6 vitamer preferably pyridoxine and pyridoxamine followed by
5) administering an IV infusion containing about 375 mg/m² of 5-FU over a period of 22 hours,

In some embodiments, the combination, or compositions of the present invention for its use according to the above in the treatment of cancer, in particular breast cancer and colorectal cancer including advanced and metastatic colorectal cancer and pancreas adenocarcinomas are administered sequentially or concomitantly with one or more therapeutic active agent such as to anti-cancer compound, chemotherapeutic or radiotherapeutic agents.

The term “anti-cancer compound” has its general meaning in the art and refers to anti-cancer compounds used in anti-cancer therapy such as tyrosine kinase inhibitors, tyrosine kinase receptor (TKR) inhibitors, EGFR tyrosine kinase inhibitors, anti-EGFR compounds, anti-HER2 compounds, Vascular Endothelial Growth Factor Receptors (VEGFRs) pathway inhibitors, interferon therapy, alkylating agents, anti-metabolites, immunotherapeutic agents, Interferons (IFNs), Interleukins, and chemotherapeutic agents such as described below.

The term “tyrosine kinase inhibitor” or “TKI” has its general meaning in the art and refers to any of a variety of therapeutic agents or drugs such as compounds inhibiting tyrosine kinase, tyrosine kinase receptor inhibitors (TKRI), EGFR tyrosine kinase inhibitors, EGFR antagonists. The term “tyrosine kinase inhibitor” or “TKI” has its general meaning in the art and refers to any of a variety of therapeutic agents or drugs that act as selective or non-selective inhibitors of receptor and/or non-receptor tyrosine kinases. Examples of tyrosine kinase inhibitors and related compounds suitable for use in methods of embodiments of the present invention include, but are not limited to Erlotinib, sunitinib (Sutent; SU11248), dasatinib (BMS-354825), PP2, BEZ235, saracatinib, gefitinib (Iressa), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Other examples of such inhibitors include, but are not limited to Erlotinib, Gefitinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

EGFR tyrosine kinase inhibitors as used herein include but are not limited to compounds selected from the group consisting of but not limited to Erlotinib, lapatinib, Rociletinib (CO-1686), gefitinib, Dacomitinib (PF-00299804), Afatanib, Brigatinib (AP26113), WJTOG3405, NEJ002, AZD9291, HM61713, EGF816, ASP 8273, AC 0010. Examples of antibody EGFR inhibitors include Cetuximab, panitumumab, matuzumab, zalutumumab, nimotuzumab, necitumumab, Imgatuzumab (GA201, R05083945), and ABT-806.

In some embodiments, the combination or composition of the present invention is administered with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a camptothecin (including the synthetic analogues topotecan or irinotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylamine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®) and doxetaxel (TAXOTERE®), chlorambucil; gemcitabine; 6-thioguanine; mercaptopurine; methotrexate; platinum analogs such as cisplatin and carboplatin; oxaliplatin (1-OHP, Oxalato-platinum Eloxatin®), vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, combination or composition of the present invention is administered with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapiesNon-specific immunotherapeutic agents are generally classified as cytokines or non-cytokine adjuvants. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). In addition to having specific or non-specific targets, immunotherapeutic agents can be active, i.e., stimulate the body's own immune response, or they can be passive, i.e. comprise immune system components that were generated external to the body. Passive specific immunotherapy typically involves the use of one or more monoclonal antibodies that are specific for a particular antigen found on the surface of a cancer cell or that are specific for a particular cell growth factor.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies anti-BAFF-R antibodies (e.g. Belimumab), anti-APRIL antibodies (e.g. anti-human APRIL antibody), and anti-IL-6 antibodies [e.g. previously described by De Benedetti et al., J Immunol (2001) 166: 4334-4340 and by Suzuki et al., Europ J of Immunol (1992) 22 (8) 1989-1993. The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most preferably the subject's own cells that were earlier isolated from a blood or tumor sample and activated (or “expanded”) in vitro. Examples of stimulatory checkpoint that may be used in the treatment of cancer include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, LAG-3, TIM-3 and VISTA.

In some embodiments, the combination or composition of the present invention is administered with a radiotherapeutic agent. The term “radiotherapeutic agent” as used herein, is intended to refer to any radiotherapeutic agent known to one of skill in the art to be effective to treat or ameliorate cancer, without limitation.

As used herein, the term “radiotherapy” for “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g., X-rays.

Further therapeutic active agent can be an antiemetic agent. Suitable antiemetic agents include, but are not limited to, metoclopramide, domperidone, prochlorperazine, promethazine, chlorpromazine, trimethobenzamide, ondansetron, granisetron, hydroxyzine, acetylleucine, alizapride, azasetron, benzquinamide, bietanautine, bromopride, buclizine, clebopride, cyclizine, dimenhydrinate, diphenidol, dolasetron, meclizine, methallatal, metopimazine, nabilone, pipamazine, scopolamine, sulpiride, tetrahydrocannabinols, thiethylperazine, thioproperazine and tropisetron. In a preferred embodiment, the antiemetic agent is granisetron or ondansetron.

In another embodiment, the further therapeutic active agent can be a hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, buprenorphine, meperidine, loperamide, ethoheptazine, betaprodine, diphenoxylate, fentanyl, sufentanil, alfentanil, remifentanil, levorphanol, dextromethorphan, phenazone, pemazocine, cyclazocine, methadone, isomethadone and propoxyphene. Suitable non-opioid analgesic agents include, but are not limited to, aspirin, celecoxib, rofecoxib, diclofenac, diflunisal, etodolac, fenoprofen, flurbiprofen, ibuprofen, ketoprofen, indomethacin, ketorolac, meclofenamate, mefenamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazepam, clorazepate, clonazepam, chlordiazepoxide and alprazolam.

In a preferred embodiment, the anti-cancer compound that can be used in addition to the fluoropyrimidine, a B6 vitamer, and a folate in the treatment of digestive cancers can be a platinum coordination compound like oxaliplatin (1-OHP, Oxalato-platinum Eloxatin®) and/or a camptothecin derivative like topotecan or irinotecan and/or an anti-angiogenic compound like bevacizumab (Avastin®).

In one embodiment, said additional active compounds may be contained in the same composition or administrated separately.

### LIST OF FIGURES

FIG. 1 is a table showing the characteristics of patients with advanced breast carcinoma treated with regimens including 5-fluorouracil, folinic acid and pyridoxine in tandem.
1 The patient categories are as follows: I, previously untreated patients whose tumours do not overexpress HER2 (1-12); II, previously untreated patients whose tumours overexpress (3+) HER2 (13-18); and III, previously treated chemotherapy patients whose tumours do not overexpress HER2 (19-27).
2 Patient with previous mastectomy.
3LC, lobular carcinoma; DC, ductal carcinoma; EE, Elston-Ellis pathology grade; Ki67 expression as percentage of cancer cells; ER, estrogen receptor; PR, progesterone receptor.
4 Axillary refers to metastatic lymph nodes in the axilla contralateral to the primary cancer.

FIG. 2 is a table showing treatment outcomes in patients with advanced breast carcinoma treated with protocols including 5-fluorouracil, folinic acid and pyridoxine in tandem.
1 Categories include I, previously untreated patients whose tumours do not overexpress HER2 (1-12); II, previously untreated patients whose tumours overexpress (3+) HER2 (13-18); and III, previously treated patients whose tumours do not overexpress HER2 (19-27)
2. The composition of the regimens is described in the text.
3. Median dose of PN prior to each injection of FUra and FA (x103 mg/day).
4. Time to achieve antitumour response, i.e., a reduction in sum diameter of ≥30%.
5. Patients who achieved PR with disappearance of most metastases had RECIST values calculated by comparing size of persistent tumours at the time of evaluation with these same tumours before treatment.
6. Percentage change in the maximum standard 18FDG uptake value normalised by lean body mass (peakSUL) assessed by PET (PERCIST).
7. Pathological response was assessed by mastectomy and locoregional resection with intent to eradicate in patients with prior mastectomy.
8. Patient with prior mastectomy. 9Time to EFS in patient 24. Na, not assessed.

FIG. 3 shows the folate metabolic pathways and inhibition of thymidylate synthase by FdUMP.

Folates: H2PteGlu: 7,8-dihydrofolate; H4PteGlu: 5,6,7,8-tetrahydrofolate; CH2-H4PteGlu: 5,10-methyltetrahydrofolate; CH3-H4PteGlu: 5-methyltetrahydrofolate; CH+-H4PteGlu: 5,10-methenyltetrahydrofolate; 10-HCO-H4PteGlu: 10-formyltetrahydrofolate; CHNH-H4PteGlu: 5-formiminotetrahydrofolate; [6S]-5-HCO-H4PteGlu: 5-formyltetrahydrofolate (FA; [6S]-leucovorin)

Enzymes: TS, thymidylate synthase; SHMT, serine hydroxymethyltransferase (PLP-dependent enzyme, including cytoplasmic SHMT1 and mitochondrial SHMT2 isoforms); GCS, glycine cleavage system (mitochondria). Other compounds and substances involved in TS inhibition: dUMP, deoxyuridylate; dTMP, thymidylate; L-HCy, L-homocysteine; L-Met, L-methionine; FUra, 5-fluorouracil; FdUMP, fluorodeoxyuridylate; [FdUMP-TS-CH2-H4PteGlu], the ternary complex resulting in TS inhibition.

FIG. 4 shows the erythrocyte pharmacokinetics of vitamin B6 after parenteral administration of high dose pyridoxine or pyridoxamine in mice

Erythrocyte pyridoxamine 5′-phosphate (PMP) and pyridoxal 5′-phosphate (PLP) levels were measured in mice after intraperitoneal administration of high doses of pyridoxine (PN) or pyridoxamine (PM). BALB/c mice (two animals per time point) were given PN or PM at 450 mg/kg at time 0 only, or twice at time 0 and after 12 hours of initiation. For each non-phosphorylated B6 vitamin explored, PMP and PLP measurements were made at 1, 3, 6, 12 and 24 hours from the start of the experiment. The table below shows the maximum concentration levels and the area under the concentration-time curve of PLP and PMP for 12 hours post-injection.

TABLE 1

| | | |
|---|---|---|
| Intracellular | | |
| Pharmacokinetics | PN, 450 mg/kg | PM, 450 mg/kg |

| Parameter | PMP | PLP | PMP | PLP |
|---|---|---|---|---|
| Mean peak | 1,961 | 637 | 4,829 | 2,326 |
| concentration of | | | | |
| vitamin B6 (nmol/L | | | | |
| cells) | | | | |
| AUC 12 h of B6 | 11,597 | 2,231 | 19,356 | 14,988 |
| vitamer (nmol/L | | | | |
| cells · 12 h) | | | | |

FIG. 5 represents the quantification of clinical and metabolic response in patients with advanced breast carcinoma treated with regimens including FUra, FA and pyridoxine in tandem.

Patients in the abscissa include I, previously untreated patients whose tumours did not overexpress HER2 (1-12); II, previously untreated patients whose tumours overexpressed (3+) HER2 (13-18); and III, previously treated patients whose tumours did not overexpress HER2 (19-27). In patients with a high target count who achieved a partial response with disappearance of most metastases, calculations of percentage reduction in sum of diameters (RECIST) were performed by comparing the size of the remaining images at the time of evaluation with the same images of tumours present before treatment. Metabolic response was assessed by the percentage change in the value of the maximum standard uptake of 18FDG normalised by lean body mass (SULpeak) obtained by PET scans (PERCIST). The dashed line at −30% represents the borderline between no change and antitumour response. Three responders were not assessed by either method (na in the histograms); one of these (12) had rapid tumour progression preventing further assessment; one patient (19) had no anatomically measurable tumour, and the other (18) had a PS 4 at presentation preventing initial assessment.

FIG. 6 represents the variation in plasma tumour marker levels in patients with advanced breast carcinoma treated with regimens including FUra, high-dose folinic acid and tandem pyridoxine.

The circles in the diagram represent the change in plasma tumour markers on treatment as a ratio of the baseline to the endpoint at the time of assessment of antitumour activity. Only patients with plasma tumour markers whose baseline concentrations were ≥two times the upper limit of normal values are shown. Open circles indicate patients whose markers reached levels at or below the upper limits of normal values. Solid circles indicate patients whose marker levels decreased but remained above the upper limit of normal values. Patient groups on the x-axis include I, previously untreated patients whose tumours do not overexpress HER2; II, previously untreated patients whose tumours overexpress (3+) HER2; and III, previously treated patients whose tumours do not overexpress HER2.

FIG. 7 represents the chronological sequence of events in 27 patients with advanced breast carcinoma treated with regimens including FUra, folinic acid and pyridoxine in tandem.

Patient groups on the ordinate include I, previously untreated patients whose tumours do not overexpress HER2 (1-12); II, previously untreated patients whose tumours overexpress (3+) HER2 (13-18); and III, previously treated patients whose tumours do not overexpress HER2 (19-27). Patients are numbered in the same order as in FIGS. 1 and 2 and FIG. 5. The bars represent progression-free survival (PFS) in all cases except for patient 24 where they represent event-free survival (EFS). The bold black lines in the bars represent the duration of treatment with FUra, folinic acid and pyridoxine in tandem, and the arrows indicate the treatment in progress at the time of this assessment. Solid squares indicate the time at which a response to treatment was recorded, i.e. a reduction in sum diameters of ≥30%. Solid circles represent the time at which mastectomy or eradicative surgery was performed. The open triangle indicates the time at which the event led to exit from the study in a single patient with a persistent response to treatment. Solid triangles indicate the time of occurrence of tumour progression in patients with a prior response to treatment.

### EXAMPLES

### Materials and Methods

The study was approved by the Medical Oncology Department board in Paul-Brousse Hospital, Assistance Publique-Hôpitaux de Paris, University Paris-Saclay. It was conducted in accordance with the basic principles of the Declaration of Helsinki. All the patients were informed of the rationale, potential benefits, and risks of the treatment. Written informed consent to study participation was obtained from all patients.

### Patients

Patients with ductal or lobular adenocarcinoma of the breast in advanced stages carrying poor prognostic features who did not receive prior chemotherapy as well as those who had previously received no more than two prior lines of chemotherapy were included. Previously treated patients could have received one chemotherapy regimen as pre-operative (neo adjuvant) or post-operative adjuvant treatment, and/or one first-line treatment for advanced disease. Prior chemotherapy had to be terminated at least 3 months before entering the study. Previous hormone therapy of any type was admitted. Owing to the great extent of tumor at presentation and poor performance status in many, patients could not be eligible neither for surgery or radiotherapy with eradication intent, nor for any available investigational therapy.

Twenty-seven patients aged 37 to 75 years old (median, 50 years) were included. Of 18 patients who had not received prior chemotherapy, 6 had tumors that overexpressed (3+) the Human Epidermal Growth Factor Receptor-2 (Her2/neu; HER2) as assessed by immunohistochemistry, and 12 had tumors that did not. Of these 18 patients, 9 presented with locally advanced unresectable tumor accompanied with distant nodal, bone, soft tissue, and/or visceral metastases, and 9 had inflammatory carcinoma of who 6 had distant metastases as well; AJCC anatomic stages were IIIB, IIIC, and IV in 2, 1, and 15 patients, respectively. Two of the 12 patients with tumors that did not overexpress HER2 who had not received prior chemotherapy presented with skin permeation nodules in one patients and skin permeation nodules, muscle invasion and ipsilateral axillary lymphadenopathy in the other, developed in the anatomical area of prior exclusive mastectomy performed 1 and 21 years before relapse. Of the 18 previously untreated patients, 15 had tumors that expressed ERs, and 3 had tumors that did not (Table 1). Nine patients with stage IV breast carcinoma diagnosed 1.5 to 25 years (mean, 8.4 years) before entering the present study had received prior chemotherapy. Of these, 4 have had prior neo adjuvant or adjuvant chemotherapy only, 3 had first-line chemotherapy for advanced disease only, and two patients had both, neo adjuvant or adjuvant chemotherapy with subsequent first-line chemotherapy for treatment of metastatic disease. All nine previously treated patients have had prior anthracycline-containing chemotherapy and 8 had taxanes as well. Eight of these 9 patients had also received FUra as part of their previous regimens of chemotherapy, including 4 who had FUra combined with folinic acid. None of them had tumors with HER2 overexpression. Of the 9 patients, 6 had tumors that expressed ERs, and 3 had said triple negative carcinoma. In addition to prior chemotherapy, the six previously treated patients whose tumors expressed ERs had previous endocrine therapy in various forms. Eight of 9 patients who had received prior chemotherapy presented with measurable tumor consisting in distant nodal, bone, soft tissue and/or visceral metastases, including one patient who had also locally advanced disease. One patient had bone metastases only. All patients had prior mastectomy (Table 1).

High initial plasma tumor marker levels (≥twice the upper limit value) were found in 15 patients who had elevated CA15-3, together with high CEA, and/or CA125 levels in 7 patients, and 8 patients, respectively. Great tumor burden was recorded in most patients (Tables 1 and 2). Eastern Cooperative Oncology Group (ECOG) performance status (PS) scores at presentation were 0-1, 2, and 3-4 in 12, 5, and 10 patients, respectively (Table 1). Two patients carried deleterious germline BRCA2 gene mutations.

### Treatment

Patients received induction regimens of chemotherapy comprising a combination of FUra and FA employed in our standard practice that were indicated for treatment of their disease and specific clinical condition, supplemented with pyridoxine in high doses accompanying each administration of FA plus FUra (Table 2). Four different combination regimens were used either alone or in sequence as indicated according to each category and/or clinical specificities of patients. Regimens were (a) FAC, consisting in one-day courses every 21 days of doxorubicin, 40 mg/m² iv Day 1; cyclophosphamide, 500 mg/m² iv Day 1; FUra, 500 mg/m² iv in 2 hours; and folinic acid (FA; [6R,S]-5-formyl tetrahydropteroylglutamate; [6R,S]-5-HCO-H4PteGlu, 200 mg/m² iv in 15′ Day 1; (b) AVCF, consisting in four-day courses every 21 days of doxorubicin, 40 mg/m² iv Day 1; vinorelbine, 25 mg/m² iv Day 1; cyclophosphamide, 250 mg/m²/day iv Days 1-4; FUra, 400 mg/m²/day iv in 2 hours, Days 1-4; and FA, 200 mg/m²/day iv in 15′ Days 1-4; (c) TCbF, consisting of four-day courses every 21 days of paclitaxel, 175 mg/m² iv Day 1; carboplatin, AUC=5 mg/ml-min iv Day 1; FUra, 400 mg/m²/day iv in 2 hours, Days 1-4; and FA, 200 mg/m²/day iv in 15′ Days 1-4; and (d) VCbF consisting of four-day courses every 21 days of vinorelbine, 25 mg/m² iv Day 1; carboplatin, AUC=5 mg/ml-min iv Day 1; FUra, 400 mg/m²/day iv in 2 hours, Days 1-4; and FA, 200 mg/m²/day iv in 15′ Days 1-4. All treatment courses were accompanied by granulocyte colony-stimulating factor (G-CSF) beginning the first day of each interval between courses.

Doxorubicin comprised in FAC and AVCF regimens was suspended in case of ≥10% decrease in left ventricular ejection fraction from baseline value. Paclitaxel included in the TCbF regimen was suspended when symptoms of sensory peripheral neuropathy (SPN) consisting in permanent hypoesthesia, paresthesia and/or dysesthesia of any intensity, and/or limb pain were first recorded. Vinorelbine-containing chemotherapy (i.e., VCbF) was indicated instead of TCbF for patients who had severe hematopoietic impairment or prior taxane-induced toxicity and was used in substitution for TCbF in cases of paclitaxel-induced SPN occurring in patients throughout the study.

Of twelve patients who were not previously treated and whose tumors did not overexpress HER2, 8 received an initial sequence of anthracycline-containing chemotherapy (4-6 courses), and then a sequence of TCbF followed by VCbF in substitution to TCbF when needed for the masons above. Anthracycline-containing chemotherapy was AVCF in 6 patients and FAC in 2. AVCF and FAC were avoided in 4 patients owing to hematologic impairment in 2 (one patient had myeloproliferative disorder and the other had profound myeloid cytopenia due to extensive bone marrow metastases and autoimmune thrombocytopenia), and to mild cardiorespiratory dysfunction in two patients; these 4 patients received taxane- and vinorelbine-containing regimens only (Table 2). The six patients who had not been previously treated, and whose tumors overexpressed HER2 (3+) received TCbF combined with the anti Her2/neu humanized monoclonal antibodies trastuzumab (6 mg/kg iv every 21 days), and pertuzumab (420 mg/patient iv every 21 days). Induction chemotherapy for the nine patients who had been previously treated consisted in TCbF in seven patients, and VCbF in two (Table 2). Premenopausal patients whose tumors expressed ERs, received long-term luteinizing hormone-releasing hormone analog (LHRHa).

Vitamin B6 is the generic name that encompasses six interconvertible compounds (i.e., B6 vitamers), namely pyridoxine (PN); pyridoxamine (PM); pyridoxal (PL); and their respective 5′ phosphorylated forms, pyridoxine 5′-phosphate (PNP), pyridoxamine 5′-phosphate (PMP), and the cofactor pyridoxal 5′-phosphate (PLP).^{27,32,33} Pyridoxine hydrochloride, the only available marketed parenteral B6 vitamer for clinical use (in 250 mg vials) was injected iv in 30′ preceding each injection of FA and FUra for a number of days defined by the schedule of the regimen used (i.e., single day in the FAC regimen, and 4 consecutive days in AVCF, TCbF, and VCbF regimens). Based on the pharmacokinetic data obtained in mice,³⁰ using approximate factors for converting doses in man from mouse data,⁴³ the daily dose of pyridoxine was augmented in patients over the duration of the present study from 1,000 mg/day to a maximum of 3,000 mg/day. The latter corresponds approximately to the high dose of PM and PN of 450 mg/kg explored in mice as described above;³⁰ in these animals, it resulted in rise of intracellular concentrations of PLP to peak levels within the range of most reported K_{d} values for binding of PLP to apo SHMT, the requirement that supports the rationale underlying the present clinical study (FIG. 2).²²⁻²⁵ The first starting dose of PN accompanying each administration of FUra and FA was 1,000 mg/day. Then, we practiced stepwise intra patient dose escalation of pyridoxine by increments of 500 to 1,000 mg/day in subsequent courses. In absence of any form of toxicity seeming attributable to the PN recorded in prior patients, the starting daily dose of PN in next patients was increased to 2,000 mg/day, and then to a maximum of 3,000 mg/day (Table 2).

In all three categories of patients described above, courses of TCbF were not limited in number a priori, and were being substituted by VCbF in cases of paclitaxel induced SPN (Table 2). Courses were repeated until antitumor response of estimated maximum degree was attained in a personalized way according to patient's condition, tolerance to treatment, phenotypic tumor specificities, and decisions from referring oncologists and clinical meetings. Several patients who achieved either a complete or a partial response of great magnitude allowing resection of residual tumor, were proposed to be subjected to mastectomy or to locoregional resection with eradication intent, after which a supplementary but limited number of postoperative courses was administered and then chemotherapy was discontinued. Once chemotherapy was terminated, patients whose tumors expressed ERs received long-term aromatase inhibitor therapy accompanied with LHRHa in premenopausal patients, and those whose tumor overexpressed HER2 received three-weekly trastuzumab during one supplementary year. Progression- and event-free survival data for each patient are indicated (Table 2 and FIG. 5). Owing to the variable post-induction therapies received by patients, we mainly focused on magnitude and characteristics of antitumor responses achieved with regimens including FUra, folinic acid and pyridoxine in tandem to assess for their antitumor potency.

### Results

Antitumor response was assessed by studying variation of the sum of diameters of anatomically measurable tumors according to Response Evaluation Criteria in Solid Tumors (RECIST) accompanied with follow-up of non-measurable tumor involvement, and that of peak standard ¹⁸FDG uptake value normalized by lean body mass (SULₚₑₐₖ) of targets as measured by Positron Emission Tomography (PET) scan according to PET Response Evaluation Criteria in Solid Tumors (PERCIST),⁴⁹ together with periodic clinical examination and measurement of plasma tumor markers. Assessment of pathologic response (TNM AJCC staging) was performed in several complete responders and in patients who had achieved partial responses of high magnitude who were subjected to mastectomy or to locoregional resection with eradication intent. Focal pathologic assessment was also obtained in several patients by imaging-oriented percutaneous biopsy of previously involved sites with persisting abnormal images after treatment.

Of 27 patients included, 26 responded to therapy and one had progressive disease. Induction treatment resulted in antitumor responses of early onset and great magnitude.

### Patients Who had not Received Prior Chemotherapy Whose Tumors Did not Overexpress HER2.

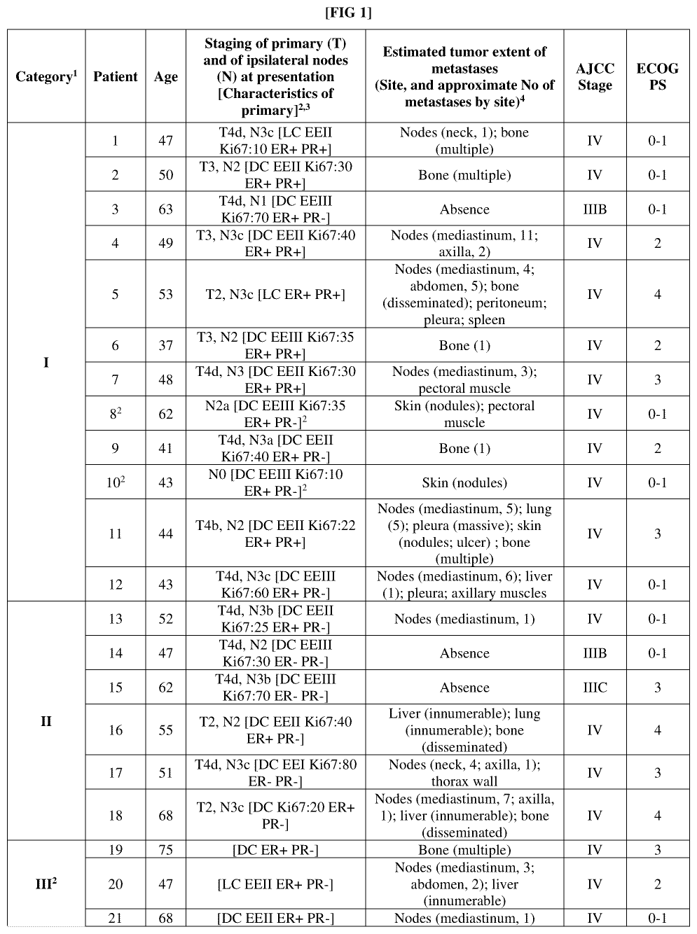

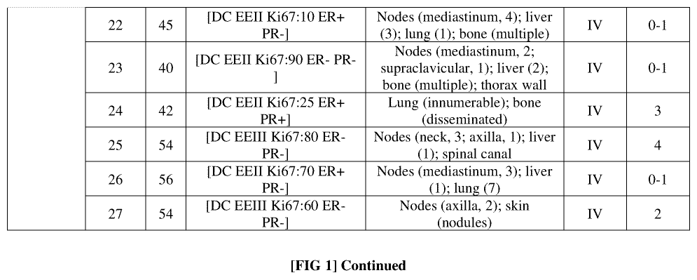

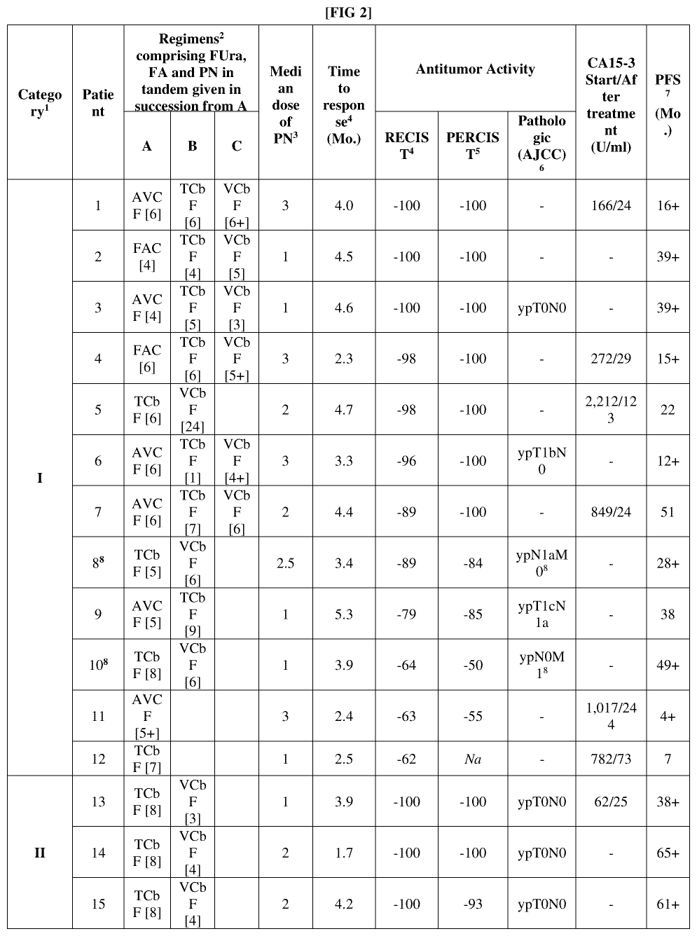

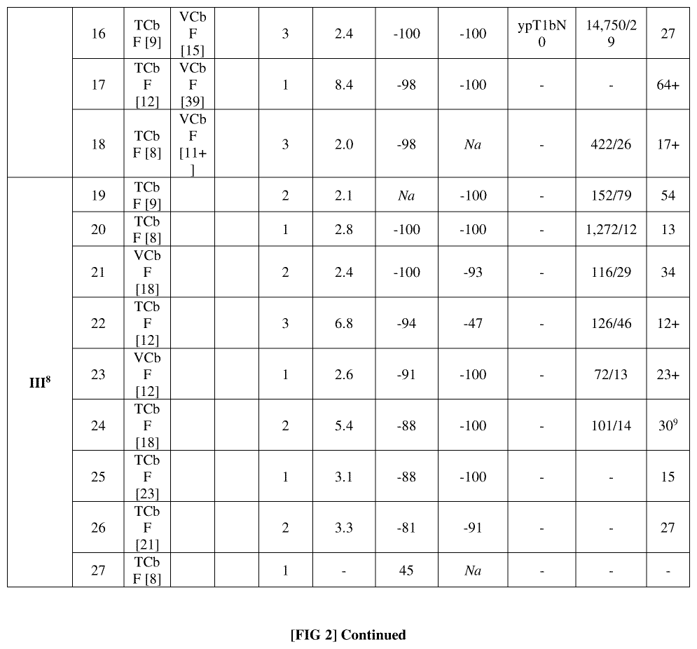

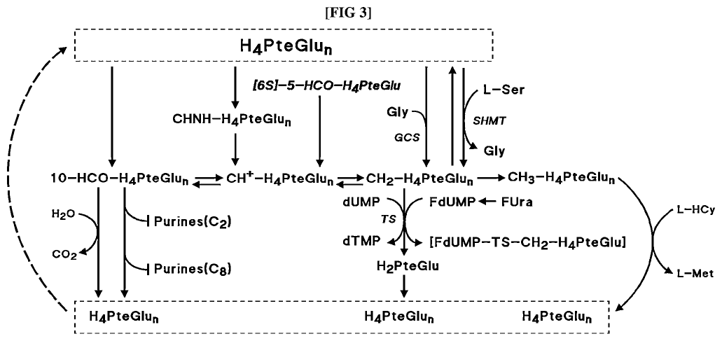

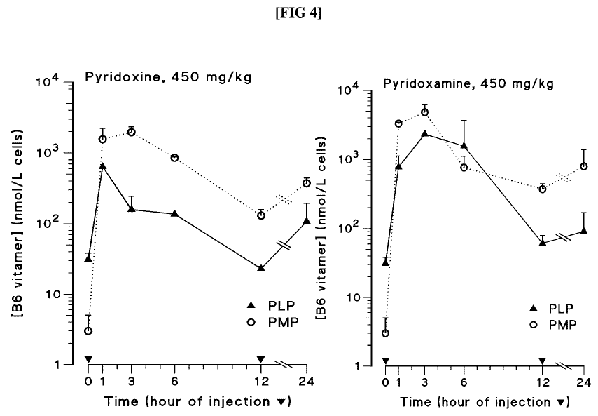

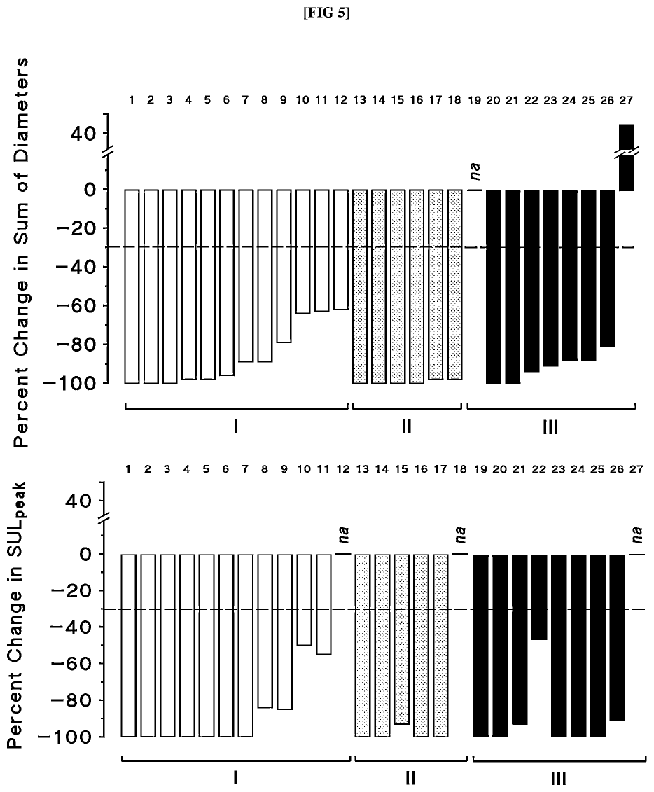

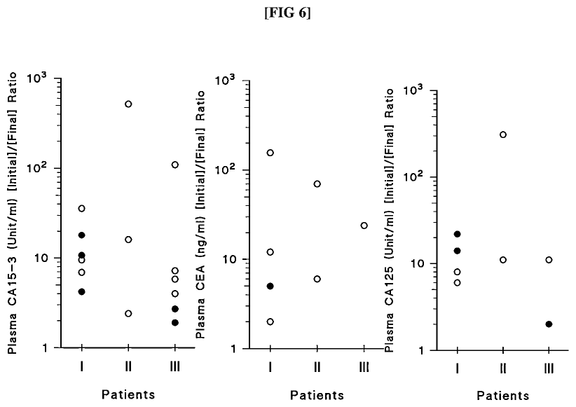

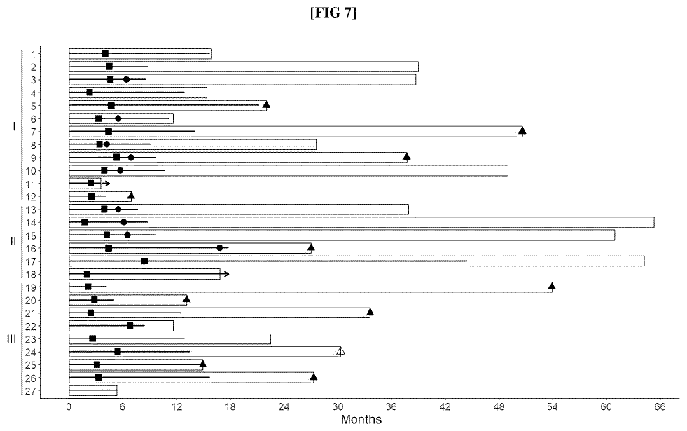

Twelve patients were included in this group. Ten patients were included at initial diagnosis and 2 patients were treated for relapse that occurred 1 year (Patient 10), and 21 years (Patient 8) after exclusive mastectomy (Table 1). Of the 12 patients, 3 attained clinical CRs and 9 had PRs with great tumor reduction rates (percent reduction in sum of longest diameters were 98, 98, 96, 89, 89, 79, 64, 63, and 62 percent), accompanied by disappearance of distant metastases that were present before treatment in 9 patients (Tables1, Table 2, and FIG. 3). Decrease of plasma CA15-3 levels from start of treatment by 4- to 36-fold occurred in the 6 patients who had elevated marker initially, of whom 3 attained normal levels (Table 2 and FIG. 4). Of 11 patients who were assessed by PET scan, 7 achieved metabolic CRs and 4 patients had metabolic PRs with reduction of SULₚₑₐₖ value by 85, 84, 55, and 50 percent. Pathologic responses were assessed by mastectomy or by resection with eradication intent in 5 patients. Of three patients who underwent mastectomy, one (Patient 3) with clinical and metabolic CR had pCR (ypTONO), one (Patient 6) with clinical reduction by 96%, and metabolic CR had minimal residual primary staged ypT1bN0, and one (Patient 9) with clinical PR (79% reduction) and metabolic PR (85% reduction) had limited residual tumor staged ypT1cN1a. Two patients with prior radical mastectomy who attained clinical and metabolic PRs of axillary lymphadenopathy and skin permeation nodules in one (Patient 8), and of permeation nodules in the second (Patient 10), had subsequent resection of soft tissue in the site of mastectomy and lymphadenectomy with eradication intent; small amounts of residual tumor <1 cm in total diameter were resected in both patients whose tumor targets had mostly disappeared. In addition, one responder with 98% reduction in tumor diameter and metabolic CR (Patient 4) had no residual breast tumor, and one partial responder (Patient 7) had minimal persistent breast tumor infiltration, as assessed by percutaneous biopsy of residual images. Of the 12 patients, 4 had relapsed after progression-free survival (PFS) times of 7, 22, 38, and 51 months, and the other eight had PFS times of 4+, 12+, 15+, 16+, 28+, 39+, 39+, and 49+ months from start of therapy (Table 2, FIG. 3 and FIG. 5).

### Patients Who had not Received Prior Chemotherapy Whose Tumors Overexpressed HER2.

Of the 6 patients included in this group, four attained clinical CRs, and 2 had PRs with percent reduction in sum of diameters by 98% in both; responses were accompanied by disappearance of distant metastases that were present before treatment in 4 patients. Decrease of plasma CA15-3 levels by 2.4- to 518-fold occurred in 3 patients who had elevated markers initially, whose levels became normal in all three (Table 2 and FIG. 4). Of five patients who were assessed by PET scan, 4 attained metabolic CRs and one had metabolic PRs with reduction of SULₚk value by 93%. Pathologic response was assessed by mastectomy in 4 clinical complete responders. Pathologic CRs staged ypTONO were attained by 3 patients who had reduction of SULₚₑₐₖ by ≥93% (Patients 13-15), and one patient with metabolic CR had residual invasive and intraductal primary staged ypT1bN0 that did not overexpress HER2 anymore (Patient 16). In addition, one responder with 98% reduction in tumor diameter and metabolic CR (Patient 17) had no residual breast and node tumor as assessed by percutaneous biopsy of residual images. Of the six patients, one had relapsed after PFS time of 27 months, and the other five had PFS times of 17+, 38+, 61+, 64+, and 65+ months from start of therapy (Table 2, FIG. 3 and FIG. 5).

### Patients Who had Received Prior Chemotherapy.

Of 9 patients in this group, 8 responded to therapy and one had progressive disease. Of 7 responders with clinically measurable disease, 2 attained clinical CRs, and 5 had PRs with tumor reduction in sum of longest diameters by 94, 91, 88, 88, and 81% (Table 2 and FIG. 3). One patient with clinically non-measurable tumor who had only bone metastases experienced dense mineralization of all initially lytic foci, together with metabolic CR (Patient 19). Responses were accompanied by disappearance of most distant metastases that were present in 7 patients before treatment. Decrease of plasma CA15-3 levels by 2- to 110-fold from baseline occurred in 6 patients who had elevated markers initially, of whom 4 attained normal levels (FIG. 4). Of eight patients who were evaluated by PET scan, 5 attained metabolic CRs and 3 had metabolic PRs with reduction in SULₚₑₐₖ values by 93, 91, and 47 percent. All responders to therapy had prior mastectomy; none were assessed for pathologic response. Of the eight responders, 5 have relapsed after PFS times of 13, 15, 27, 34, and 54 months from start of therapy, and one patient with prior genotoxic chemotherapy and radiotherapy for childhood's Ewing's Tumor, and later for post-operative treatment of breast carcinoma, had fatal AML diagnosed 18 months after completion of the present induction treatment for breast cancer. At diagnosis of AML, the patient had no progression of breast carcinoma; event-free survival (EFS) time was 30 months (Patient 24). At the time of the present report, 2 partial responders had their induction treatment terminated with no sign of disease progression; PFS times were 12+, and 23+ months (Table 2, FIG. 3 and FIG. 5).

Of twenty-six responders, 23 had clinical evaluation of response (RECIST) together with PET scan assessment (PERCIST). Conformity in percent reduction as assessed by both methods was found in 7 (30%) patients who attained clinical CRs (FIG. 3, and Table 2); moderate disparity in magnitude of response by <15 percent reduction rate (mean difference in percent reduction, 8%) was found in 2 patients with clinical CRs and in 13 with clinical PRs (65%); and disparity by 47% reduction was recorded in 1 patient with clinical PR (Patient 22). The difference in the latter consisted in persisting high ¹⁸FDG uptake in a single bone metastatic site that was mineralized under treatment, while all the other metastatic foci had FDG uptake indistinguishable from surrounding background. Of the three responders who were not evaluated by both methods, one partial responder (Patient 12) had rapidly growing tumor progression preventing further assessment, one metabolic complete responder (Patient 19) had no clinically measurable tumor at presentation, and one complete responder (Patient 18) had PS 4 at presentation preventing completion of initial assessment; the latter had a normal PET scan at the time of final evaluation. All 11 responders who had bone metastases experienced osseous remineralization, and serosal effusion disappeared in 3 patients who had pleural involvement. Antitumor responses were rapidly attained (Table 2); approximate times required for achieving an objective response from start of treatment ranged from 1.7 to 8.4 months (median, 3.4 months). Responses were accompanied by disappearance of tumor related symptoms in all cases.

Assessment of toxicity before initiation of each cycle of therapy did not record neither any form of unusual toxicity nor toxic effect of greater magnitude than that expected with each regimen used. Interruption of paclitaxel due to sensory peripheral neuropathy occurred in 11 out of 24 patients (46%) who were treated with the TCbF regimen at cumulative amounts of paclitaxel ranging from 156 to 2833 mg/m² (mean, 1604 mg/m²), including a single patient who had severe SPN with limb pain, and dysesthesia after the first course of TCbF. Patients with paclitaxel induced neuropathy, whose chemotherapy was either interrupted or pursued with VCbF had further progressive decrease of neurologic symptoms, and then disappearance occurring in most patients during follow-up. The 13 patients who did not develop SPN under TCbF received smaller mean cumulative amount of paclitaxel (mean, 983 mg/m²; range 570-1240 mg/m²) than that received by patients with neuropathy. The 2 patients whose chemotherapy included VCbF without any prior paclitaxel did not develop SPN. Except for interruption of paclitaxel as described above, no dose reductions of any cytostatic agent or increasing intervals between courses due to unusual or unexpectedly excessive hematologic and/or visceral toxicity were required. The single case of acute myelogenous leukemia carrying the 17q-chromosome aberration marker occurred 18 months after cessation of induction treatment in one patient who had prior genotoxic cytostatics and radiotherapy.

### DISCUSSION

Clinical signification of antitumor potency of chemotherapy with regard to long-term outcome for patients with breast carcinoma in advanced stages, an efficiently treatable but essentially incurable condition, is a difficult, and amply debated issue. Combination chemotherapy regimens showed a statistically significant advantage over any single agent therapy with regard to antitumor response, time to progression and survival,^{35,36} but combinations also produce more toxicity leading to detrimental effects on quality of life. Moreover, for patients unselected for phenotypic sub specificities, there is no recognized standard combination regimen among the most active ones, since taxane-containing combinations were significantly but only modestly better than anthracycline-based combinations in terms of response rate and PFS, but not for survival.³⁶ Among combination treatments, platinum derivative-containing regimens of various compositions were reported to be slightly more potent than non-platinum combination treatments regarding response rate and event-free survival, this statistically significant difference being more marked in the subset of patients with triple negative advanced breast carcinoma in which moderate improvement of survival was also found.⁵⁰ Remarkably, improvement of long-term prognosis related to degree of antitumor efficacy of induction treatments has been firmly demonstrated from studies of patients with high-risk localized breast carcinoma subjected to pre-operative induction treatment. Meta-analysis of a large number of studies demonstrated that patients who attained pathologic complete responses under preoperative treatment had much greater event-free and survival times than did those who had residual tumor, these statistically significant improvements being maintained over long periods of time.⁵¹ However, pathologic CRs, whose rate vary in subsets of patients with phenotypically distinct tumors, are attained by only approximately 20 percent of all patients with clinically localized breast cancer subjected to induction treatment,⁵¹ which emphasizes the need for powerful newer strategies applicable to all subgroups of patients with this neoplasm in need of chemotherapy.

In an attempt at improving the antitumor potency of chemotherapy, we explored a new method to modulate the cytotoxic activity of FUra and folinic acid included in standard regimens by adding high-dose pyridoxine accompanying each administration of FUra and FA. Response to therapy was rapidly attained by 26 out of 27 patients included in the three subsets presented herein. However, this strikingly high response rate nor the short median time of 3.4 months required to attain a response can be paired to prior therapeutic series for a comparison owing to the limited number of patients included. Antitumor responses of great magnitude were attained by all 18 previously untreated patients presenting with unresectable breast adenocarcinoma, of which most had numerous distant metastases (Table 2). Complete responses of long duration and partial responses with great tumor reduction rates were achieved by these patients, with the more marked favorable overall results attained by patients whose tumors overexpressed HER2, as expected from prior studies with patients treated with chemotherapy associated with anti Her-2/neu monoclonal antibodies, preferably with trastuzumab and pertuzumab combined.⁵² Moreover, in 6 of 12 previously untreated patients who attained a clinical response with reduction in sum of tumor diameters by 96% or more who were subjected to mastectomy (Table 2, FIG. 3), two had only small residual tumor staged ypT1bN0, and 4 had pathologic CRs. In addition, 2 other responders with tumor reduction rate of 98% in both, had no residual tumor in breast as assessed by ultrasonography-oriented biopsy of images persisting after induction treatment. Although still partial, these results outline the capacity of the treatment presented herein at achieving major tumor reduction, and eventually disease eradication in patients with previously untreated advanced inoperable and/or metastatic breast carcinoma expressing or not the ERs, either without or with HER2/neu overexpression. Complete clinical and metabolic responses of long duration and partial responses with great tumor reduction rates together with disappearance of distant metastases were also achieved by patients who had received prior treatment. Among the 8 responders, 7 had been treated previously with combination chemotherapy regimens including FUra, of whom 4 received folinic acid as well. Further studies are needed to explore whether addition to FUra of folinic acid and high-dose B6 vitamer in tandem can overcome prior resistance to the fluoropyrimidines. The present PN dose escalation pilot study does not enable correlating the magnitude of antitumor responses nor the rapidity to attain a response with the median daily dose of B6 vitamer received by each patient during the time of treatment (Table 2). Larger trials including pharmacokinetically-monitored dose finding studies are necessary to explore this issue.

Assessment of toxicity due to treatment before initiation of each cycle could not discover any form of toxicity greater than that expected with each regimen in the absence of B6 vitamer, nor any unexpected toxic effect. In particular, the use of high cumulative doses of pyridoxine was not accompanied with greater incidence, or higher grades, of sensory peripheral neuropathy than that expected with the use of paclitaxel as scheduled in the TCbF regimen. Except for one patient who had early onset acute peripheral neuropathy following the first course of TCbF, the cumulative amount of paclitaxel received by patients who experienced dose-limiting sensory peripheral neuropathy was within the range of that previously reported.^{53,54}

In addition to the strikingly high response rate reported herein, the great magnitude of antitumor responses of long duration which were rapidly attained by patients who carried in most cases great tumor burden, suggest that addition of vitamin B6 in high dose may strongly enhance the antitumor activity of standard chemotherapy combination regimens comprising FUra and FA. Magnitude of the antitumor response and how quickly responses are attained have been reported as marks of antitumor potency in solid tumors. Improvement of long-term prognosis related to early tumor shrinkage, and to depth of antitumor response under induction cytostatic treatment was reported from studies of patients with advanced stage colorectal carcinoma,^{55,56} and of patients with advanced breast carcinoma overexpressing HER2 treated with chemotherapy combined with trastuzumab.⁵⁷ In these patients, early tumor shrinkage and great magnitude of antitumor response were predictors of favorable long-term outcomes.⁵⁵⁻⁵⁷

The remarkable antitumor activity observed in the present study may represent the difference with that reported elsewhere with combination regimens administered in their standard form. Strength of antitumor activity achieved in the present study is of similar level than that recently reported in patients with advanced carcinomas of the digestive tract treated with regimens including FUra, FA, and PN.⁴⁷ Demonstration of potentiation of FUra by FA and high dose B6 vitamer in tandem requires a first step of phase II trials of combination schemas for patients with potentially FUra-sensitive tumors. Murine experiments reported herein indicate that parenteral PM carries an advantage over PN to expand intracellular PLP pools which may facilitate SHMT-dependent synthesis of CH2-H4PteGlu to improve the modulation of FUra. Exploration of these findings requires first the production and development of pyridoxamine for clinical use. Vitamin B6 pharmacokinetic studies with emphasis on intracellular PLP levels, should accompany these trials to optimize the modulation of the fluoropyrimidines in accordance with experimental data.

### REFERENCES

1. Santi, D. V., McHenry, C. S., & Sommer, H. Mechanism of interaction of thymidylate synthetase with 5-fluorodeoxyuridylate. Biochemistry. 13, 471-481 (1974)
2. Danenberg, P. V. & Danenberg, K. D. Effect of 5,10-methylenetetrahydrofolate on the dissociation of 5-Fluoro-2′-deoxyuridylate from thymidylate synthetase: evidence for an ordered mechanism. Biochemistry. 17, 4018-4024 (1978)
3. Lockshin, A. & Danenberg, P. V. Biochemical factors affecting the tightness of fluorodeoxyuridylate binding to human thymidylate synthetase. Biochem Pharmacol. 30, 247-257 (1981)
4. Ullman, B., Lee, M., Martin, D. W. & Santi, D. V. Cytotoxicity of 5-fluoro-2′-deoxyuridine: Requirement for reduced folate cofactors and antagonism by methotrexate. Proc Natl Acad Sci USA. 75, 980-983 (1978)
5. Wyatt, M. D. & Wilson, D. M. III. Participation of DNA repair in the response to 5-fluorouracil. Cell Mol Life Sci. 66, 788-799 (2009)
6. Mani, C., Pai, S., Papke, C. M., Palle, K. & Gmeiner W. H. Thymineless death by the fluoropyrimidine polymer F10 involves replication fork collapse and is enhanced by Chk1 inhibition. Neoplasia. 20, 1236-1245 (2018)
7. Cortez, D. Preventing replication fork collapse to maintain genome integrity. DNA Repair. 32, 149-157 (2015)
8. Hagenkort, A. et al. dUTPase inhibition augments replication defects of 5-Fluorouracil. Oncotarget. 8, 23713-23726 (2017)
9. Machover, D. et al. Treatment of advanced colorectal and gastric adenocarcinomas with 5-FU combined with high-dose folinic acid: a pilot study. Cancer Treat Rep. 66, 1803-1807 (1982)
10. Piedbois, P. et al. for the Advanced Colorectal Cancer Meta-Analysis Project. Modulation of 5-fluorouracil by leucovorin in patients with advanced colorectal cancer: Evidence in terms of response rate. J Clin Oncol. 10, 896-903 (1992)
11. Romanini, A. et al. Role of folylpolyglutamates in biochemical modulation of fluoropyrimidines by leucovorin. Cancer Res. 51, 789-793 (1991)
12. Wright, J. E. et al. Selective expansion of 5,10-Methylenetetrahydrofolate pools and modulation of 5-fluorouracil antitumor activity by leucovorin in vivo. Cancer Res. 49, 2592-2596 (1989)
13. Houghton J A, et al. Influence of dose of [6RS]-leucovorin on reduced folate pools and 5-fluorouracil-mediated thymidylate synthase inhibition in human colon adenocarcinoma xenografts. Cancer Res. 50, 3940-3946 (1990)
14. Houghton, J. A. et al. Relationship between dose rate of [6RS] leucovorin administration, plasma concentrations of reduced folates, and pools of 5,10-methylenetetrahydrofolates and tetrahydrofolates in human colon adenocarcinoma xenografts. Cancer Res. 50, 3493-3502 (1990)
15. Zhang, Z-G. & Rustum, Y. M. Effect of diastereoisomers of 5-formyltetrahydrofolate on cellular growth, sensitivity to 5-fluoro-2′-deoxyuridine, and methylenetetrahydrofolate polyglutamate levels in HCT-8 cells. Cancer Res. 51, 3476-3481 (1991)
16. Boarman, D. M. & Allegra, C. J. Intracellular metabolism of 5-formyl tetrahydrofolate in human breast and colon cell lines. Cancer Res. 52, 36-44 (1992)
17. Priest, D. G., Schmitz, J. C. & Bunni, M. A. Folate metabolites as modulators of antitumor drug activity in Chemistry and Biology of Pteridines and Folates (ed. Ayling, J. E.) 693-697 (Plenum Press, New York, 1993)
18. Voeller, D. M. & Allegra, C. J. Intracellular metabolism of 5-methyltetrahydrofolate and 5-formyltetrahydrofolate in a human breast cancer cell line. Cancer Chemother Pharmacol. 34, 491-496 (1994)
19. Machover, D. et al. Cytotoxic synergism of methioninase in combination with 5-fluorouracil and folinic acid. Biochem Pharmacol. 61, 867-876 (2001)
20. Wettergren Y, Taflin H, Odin E, Kodeda K & Derwinger K. A pharmacokinetic and pharmacodynamic investigation of Modufolin compared to Isovorin after single dose intravenous administration to patients with colon cancer: a randomized study Cancer Chemother Pharmacol. 75, 37-47 (2015)
21. Nixon, P. F., Slutsky, G., Nahas, A. & Bertino, J. R. The turnover of folate coenzymes in murine lymphoma cells. J Biol Chem. 248, 5932-5936 (1973)
22. Schirch, L V. et al. Serine transhydroxymethylase. Subunit structure and the involvement of sulfhydryl groups in the activity of the enzyme. J Biol Chem. 248, 6456-6461 (1973)
23. Jones, C. W. & Priest, D. G. Interaction of pyridoxal 5′-phosphate with apo-serine hydroxymethyl transferase. Biochim Biophys Acta. 526, 369-374 (1978)
24. Perry, C. Yu, S., Chen, J., Matharu, K. S. & Stover, P. J. Effect of vitamin B6 availability on serine hydroxymethyltransferase in MCF-7 cells. Arch Biochem Biophys. 462, 21-27 (2007)
25. Giardina, G., et al. How pyridoxal 5′-phosphate differentially regulates human cytosolic and mitochondrial serine hydroxymethyltransferase oligomeric state. FEBS J. 282, 1225-1241 (2015)
26. Kikuchi, G., Motokawa, Y., Yoshida, T. & Hiraga, K. Glycine cleavage system: reaction mechanism, physiological significance, and hyperglycinemia. Proc Jpn Acad Ser B Phys Biol Sci. 84, 246-263 (2008)
27. Ueland, P. M., Ulvik, A., Rios-Avila, L., Midttun, 0. & Gregory, J. F. Direct and functional biomarkers of vitamin B6 status. Annu Rev Nutr. 35, 33-70 (2015)
28. Zempleni, J. & KUbler, W. The utilization of intravenously infused pyridoxine in humans. Clin Chim Acta. 229, 27-36 (1994)
29. Martinez, M., Cuskelly, G. J., Williamson, J., Toth, J. P. & Gregory, J. F. Vitamin B-6 deficiency in rats reduces hepatic serine hydroxymethyl transferase and cystathionine β-synthase activities and rates of in vivo protein turnover, homocysteine remethylation and transsulfuration. J Nutr. 130, 1115-1123 (2000)
30. Machover, D. et al. Enhancement of 5-Fluorouracil Cytotoxicity by Pyridoxal 5′-Phosphate and Folinic Acid in Tandem. J Pharmacol Exp Ther. 366, 238-243 (2018)
31. Beechey, R. P. & Happold F. C. Pyridoxamine Phosphate Transaminase. Biochem J. 66, 520-527 (1957)
32. Ink S. L. & Henderson L V. M. Vitamin B6 metabolism. Ann Rev Nutr. 4, 455-70 (1984)
33. Di Salvo, M. L., Contestabile, R. & Safo, M. K. Vitamin B6 salvage enzymes: mechanism, structure and regulation. Biochim Biophys Acta. 1814, 1597-1608 (2011)
34. Musayev, F. N., Di Salvo, M. L. Ko, T-P, Schirch, V. & Safo, M. K. Structure and properties of recombinant human pyridoxine 5′-phosphate oxidase. Protein Sci. 12, 1455-1463 (2003)
35. Carrick, S. et al. Single agent versus combination chemotherapy for metastatic breast cancer. Cochrane Database of Systematic Reviews. 2, 1-125 (2009)
36. Piccart-Gebhart, M. J. et al. Taxanes Alone or in Combination with Anthracyclines as First-Line Therapy of Patients with Metastatic Breast Cancer. J Clin Oncol. 26, 1980-1986 (2008)
37. Margolin, K. A. et al. Effective initial therapy of advanced breast cancer with fluorouracil and high-dose, continuous infusion calcium leucovorin. J Clin Oncol. 10, 1278-1283 (1992)
38. Klaassen, U., Wilke, H. & Seeber, S. Weekly high-dose infusional 5-fluorouracil (HD-5-FU) combinations in the treatment of advanced breast cancer: results of phase I/II studies with weekly 24-hour infusion of HD-5-FU plus high-dose folinic acid (FA) alone and in combination with paclitaxel and cisplatin. J Infus Chemother. 6, 127-132 (1996)
39. Wilke, H. et al. Phase I/II study with a weekly 24-hour infusion of 5-fluorouracil plus high-dose folinic acid (HD-FU/FA) in intensively pretreated patients with metastatic breast cancer. Ann Oncol. 7, 55-58 (1996)
40. Johnson, D. H., Paul, D., & Hande, K. R. Paclitaxel, 5-fluorouracil, and folinic acid in metastatic breast cancer: BRE-26, a phase II trial. Semin Oncol. 24, 22-25 (1997)
41. Nicholson, B. et al. Paclitaxel/5-fluorouracil/leucovorin in metastatic breast cancer: a Vanderbilt Cancer Center phase II trial. Semin Oncol. 24, 11-20 (1997)
42. Yeh, K. H. et al. Phase II study of weekly vinorelbine and 24-h infusion of high-dose 5-fluorouracil plus leucovorin as first-line treatment of advanced breast cancer. Br J Cancer. 92, 1013-1018 (2005)
43. Gebbia, V. et al. Vinorelbine and 5-fluorouracil bolus and/or continuous venous infusion plus levofolinic acid as second-line chemotherapy for metastatic breast cancer: an analysis of results in clinical practice of the Gruppo Oncologico Italia Meridionale (GOIM). Anticancer Res. 26, 3143-3150 (2006)
44. Oliva, C. et al. Vinorelbine and fluorouracil plus leucovorin combination (ViFL) in patients with anthracycline and taxane-pretreated metastatic breast cancer: a phase II study. J Cancer Res Clin Oncol. 136, 411-417 (2010)
45. Schaumburg. H. et al. Sensory neuropathy from pyridoxine abuse. A new megavitamin syndrome. N Engl J Med. 309, 445-448 (1983)
46. Rimland, B., Callaway, E. & Dreyfus, P. The effect of high doses of vitamin B6 on autistic children: A double blind crossover study. Am J Psychiatry. 135, 472-475 (1978)
47. Machover, D. et al. Pharmacologic modulation of 5-fluorouracil by folinic acid and high-dose pyridoxine for treatment of patients with digestive tract carcinomas. Scientific Reports. https://doi.org/10.1038/s41598-021-92110-5 (2021)
48. Freireich, E. J., Gehan, E. A., Rall, D. P., Schmidt, L. H. & Skipper, H. E. Quantitative comparison of toxicity of anticancer agents in mouse, rat, hamster, dog, monkey, and man. Cancer Chemother Rep. 50, 219-244 (1966)
49. Pinker, K., Riedl, C. & Weber, W. A. Evaluating tumor response with FDG PET: updates on PERCIST, comparison with EORTC criteria and clues to future developments. Eur J Nucl Mol Imaging. 44, 55-66 (2017)
50. Egger, S. J., et al. Platinum-containing regimens for metastatic breast cancer. Cochrane Database of Systematic Reviews. 6, 1-130 (2017)
51. Spring, L. M. et al. Pathological complete response after neoadjuvant chemotherapy and impact on breast cancer recurrence and survival: a comprehensive meta-analysis. Clin Cancer Res. 26, 2838-2848 (2020)
52. Baselga, J. et al. for the CLEOPATRA Study Group. Pertuzumab plus trastuzumab plus docetaxel for metastatic breast cancer. N Engl J Med. 366, 109-119 (2012)
53. Ewertz, M., Qvortrup, C. & Eckhoff, L. Chemotherapy-induced peripheral neuropathy in patients treated with taxanes and platinum derivatives. Acta Oncologica. 54, 587-591 (2015)
54. Miltenburg, N. C. & Boogerd, W. Chemotherapy-induced neuropathy: A comprehensive survey. Cancer Treatment Reviews. 40, 872-882 (2014)
55. Taïeb, J. et al. Exploratory analyses assessing the impact of early tumour shrinkage and depth of response on survival outcomes in patients with RAS wild-type metastatic colorectal cancer receiving treatment in three randomised panitumumab trials. J Cancer Res Clin Oncol. 144, 321-335 (2018)
56. Heinemann, V. et al. Early tumour shrinkage (ETS) and depth of response (DpR) in the treatment of patients with metastatic colorectal cancer (mCRC). Eur J Cancer. 51, 1927-1936 (2015)
57. Che, Y-Q. et al. Depth of response and early tumor shrinkage for predicting clinical outcomes in HER2-positive metastatic breast cancer treated with trastuzumab. Cancer Manag Res. 12, 8527-8534 (2020)

1-13. (canceled)

14. A method of treating breast cancer in a subject, comprising administering to a subject in need thereof an antitumor pharmaceutical composition comprising (i) a fluoropyrimidine, (ii) a B6 vitamer, and (iii) a folate.

15. The method according to claim 14, wherein the administering of the (i) a fluoropyrimidine, (ii) a B6 vitamer, and (iii) a folate of the antitumor pharmaceutical composition is simultaneous, separate or sequential.

16. The method according to claim 14, wherein the fluoropyrimidine is selected from the group consisting of 5-fluorouracil, capecitabine, 5-fluoro-2′-deoxyuridine, ftorafur, emitefur, eniluracil/5-FU, S-1, UFT and mixtures thereof.

17. The method according to claim 14, the fluoropyrimidine is 5-fluorouracil.

18. The method according to claim 14, wherein the B6 vitamer is selected from the group consisting of pyridoxine, pyridoxal, pyridoxamine, 5′-phosphorylated derivatives thereof, acetate esters thereof, pharmaceutically compatible salts thereof, and mixtures thereof.

19. The method according to claim 14, wherein the B6 vitamer is selected from the group consisting of pyridoxine and pyridoxamine.

20. The method according to claim 14, wherein the B6 vitamer is administered at a dose high enough to achieve plasma or intracellular levels of PLP equal to or greater than those required for optimal synergistic effect of fluoropyrimidines.

21. The method according to claim 14, wherein the folate is selected from the group consisting of folic acid, dihydrofolate, tetrahydrofolate, 5-methyltetrahydrofolate, 5,10-methylenetetrahydrofolate, 5,10-methenyltetrahydrofolate, 5-formiminotetrahydrofolate, 10-formyltetrahydrofolate, [6RS]-5-formyltetrahydrofolate, the active stereoisomers of any of these folates, [6R]-5,10-methylenetetrahydrofolate, [6S]-5-formyltetrahydrofolate, pharmaceutically compatible salts thereof, and mixtures thereof.

22. The method according to claim 14, wherein the folate is 5-formyltetrahydrofolate, [6S]-5-formyltetrahydrofolate or the calcium or sodium levofolinate.

23. The method according to claim 14, wherein said composition is administered sequentially or concomitantly with one or more immunotherapeutic, chemotherapeutic or radiotherapeutic agent.

24. A method of treating cancer in a subject, comprising administering to a subject in need thereof an antitumor pharmaceutical composition comprising (i) a fluoropyrimidine, (ii) a B6 vitamer, and (iii) a folate, said treatment comprising the following steps:
on day 1,
a) administering over a period of 10-30 minutes, preferably about 15 minutes, an IV bolus containing 100-1000 mg/m², preferably 200 mg/m² of a folate, preferably levofolinate or arfolitixorin followed by
b) administering over a period of 10-30 minutes, preferably about 15 minutes, an IV bolus, containing a dose higher than 3000 mg, of a B6 vitamer preferably pyridoxine or pyridoxamine followed by
c) administering an IV bolus containing 100-1000 mg/m², preferably about 600 mg/m² of 5-FU over a period of 2 hours optionally followed by
d) administering an IV infusion containing 100-500 mg/m², preferably about 350 mg/m² of 5-FU over a period of 22 hours,
Said steps a) to d) are repeated on day 2,
wherein, optionally:
1) on day 1, said steps a) to d) are preceded by:
i) administering over a period of 30-180 minutes, preferably 120 minutes an IV bolus containing 50-100 mg/m², preferably about 85 mg/m² of oxalato-platinum (1-OHP) optionally followed by
ii) administering over a period of 10-60 minutes, preferably about 30 minutes an IV bolus containing 100-200 mg/m², preferably about 180 mg/m² of camptothecin-11 (CPT11), and
2) on day 1 and on day 2, between said steps c) and d) administering over a period of 10-30 minutes, preferably about 15 minutes, an IV bolus, containing a dose higher than 3000 mg of a B6 vitamer preferably pyridoxine and pyridoxamine.

25. A method of treating cancer in a subject, comprising administering to a subject in need thereof an antitumor pharmaceutical composition comprising (i) a fluoropyrimidine, (ii) a B6 vitamer, and (iii) a folate, said treatment comprising the following steps:
I) on day 1,
a) administering over a period of about 120 minutes an IV bolus containing about 85 mg/m² of oxalato-platinum (1-OHP, oxaliplatin) followed by
b) administering over a period of about 30 minutes an IV bolus containing about 180 mg/m² of camptothecin-11 (CPT11, Irinotecan) followed by
c) administering over a period of about 15 minutes, an IV bolus containing about 200 mg/m² of a folate, selected from levofolinate and arfolitixorin followed by
d) administering over a period of 10-30 minutes, preferably about 15 minutes, an IV bolus, containing a dose higher than 3000 mg of a B6 vitamer preferably pyridoxine and pyridoxamine followed by
e) administering an IV bolus containing about 625 mg/m² of 5-FU over a period of about 2 hours optionally followed by
f) administering a second IV bolus containing about 375 mg/m² of 5-FU over a period of 22 hours, and
on day 2,
a) administering over a period of about 15 minutes, an IV bolus containing about 200 mg/m² of a folate, selected from levofolinate and arfolitixorin followed by
b) administering over a period of 10-30 minutes, preferably about 15 minutes, an IV bolus, containing a dose higher than 3000 mg of a B6 vitamer preferably pyridoxine and pyridoxamine followed by
c) administering an IV bolus containing about 625 mg/m² of 5-FU over a period of about 2 hours optionally followed by
d) administering an IV infusion containing about 375 mg/m2 of 5-FU over a period of 22 hours,
or,
II) on day 1,
a) administering over a period of about 120 minutes an IV bolus containing about 85 mg/m² of oxalato-platinum (1-OHP, oxaliplatin) followed by
b) administering over a period of about 30 minutes an IV bolus containing about 180 mg/m² of camptothecin-11 (CPT11, Irinotecan) followed by
c) administering over a period of about 15 minutes, an IV bolus containing about 200 mg/m² of a folate, selected from levofolinate and arfolitixorin followed by
d) administering over a period of 10-30 minutes, preferably about 15 minutes, an IV bolus, containing a dose higher than 3000 mg of a B6 vitamer preferably pyridoxine and pyridoxamine followed by
e) administering an IV bonus containing about 625 mg/m² of 5-FU over a period of about 2 hours optionally followed by
f) administering over a period of 10-30 minutes, preferably about 15 minutes, a second IV bolus, containing a dose higher than 3000 mg of a B6 vitamer preferably pyridoxine and pyridoxamine followed by
g) administering a continuous infusion containing about 375 mg/m2 of 5-FU over a period of 22 hours, and
on day 2,
a) administering over a period of about 15 minutes, an IV bolus containing about 200 mg/m² of a folate, selected from levofolinate and arfolitixorin followed by
b) administering over a period of 10-30 minutes, preferably about 15 minutes, an IV bolus, containing a dose higher than 3000 mg of a B6 vitamer preferably pyridoxine and pyridoxamine followed by
c) administering an IV bolus containing about 625 mg/m² of 5-FU over a period of about 2 hours optionally followed by
d) administering over a period of 10-30 minutes, preferably about 15 minutes, a second IV bolus, containing a dose higher than 3000 mg of a B6 vitamer preferably pyridoxine and pyridoxamine followed by
e) administering an IV infusion containing about 375 mg/m² of 5-FU over a period of 22 hours,

26. The method according to claim 24, wherein the cancer is selected from the bladder, blood, bone, bone marrow, brain, breast, colon, oesophagus, gastrointestinal, gum, head, kidney, liver, lung, nasopharynx, neck, ovary, prostate, skin, stomach, testis, tongue, pancreatic or uterus cancer in particular colorectal cancer including advanced and metastatic colorectal cancer and pancreas adenocarcinomas.

27. The method according to claim 25, wherein the cancer is selected from the bladder, blood, bone, bone marrow, brain, breast, colon, oesophagus, gastrointestinal, gum, head, kidney, liver, lung, nasopharynx, neck, ovary, prostate, skin, stomach, testis, tongue, pancreatic or uterus cancer in particular colorectal cancer including advanced and metastatic colorectal cancer and pancreas adenocarcinomas.

28. The method according to claim 24, wherein the cancer is colorectal cancer including advanced and metastatic colorectal cancer and pancreas adenocarcinomas, and said treatment is administered each 14 days.

29. The method according to claim 25, wherein the cancer is colorectal cancer including advanced and metastatic colorectal cancer and pancreas adenocarcinomas, and said treatment is administered each 14 days.
